SEQUENCE LISTING (103 sequences)

SEQ ID NO: 1 - DNA, 822 bp, Rhizomucor miehei
gaagccgaagcttccatcgacggaggtattagagccgctacttctcaggaaatcaacgaacttacttactatacaactttgtcagctaattcttactgtagaactgttattcctggtgctacttgggattgcatacattgtgacgccactgaagatttaaagataattaaaacctggtctactttgatttacgacactaacgctatggttgctagaggagattccgagaagactatttatatcgtgtttagaggttcttcatctattcgtaattggatcgctgatttgacattcgttccagtctcttaccctccagtttctggtactaaggttcacaaaggatttcttgattcttatggtgaagttcaaaacgagttggttgctactgtcttggatcagtttaaacaatacccatcttataaggttgctgtcactggtcactctttgggaggtgctactgccttgctgtgtgctttagatttataccagagagaggaaggattgtcttcaagtaacctattcttgtacactcaaggtcagcctagagttggagatccagcatttgctaattatgtggtttctactggtattccatatagacgtactgttaacgaaagagacatagtaccacacttgcctccagctgccttcggatttctgcatgccggtgaagagtactggatcacagataattctcctgaaaccgttcaagtgtgtacatctgatttagagacttccgactgctctaacagtattgttccatttacttcagttcttgatcatttgtcttattttggaattaacaccggtttgtgtacttaa

SEQ ID NO: 2 - protein, 269 aa, Rhizomucor miehei
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 3 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTNDTETSDCSNSIVPFTSVLDHLSYFGINTGLCT

SEQ ID NO: 4 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSNCTNSIVPFTSVLDHLSYFGINTGLCT

SEQ ID NO: 5 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPNTTVLDHLSYFGINTGLCT

SEQ ID NO: 6 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTNDTETSDCSNSIVPNTTVLDHLSYFGINTGLCT

SEQ ID NO: 7 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSNCTNSIVPNTTVLDHLSYFGINTGLCT

SEQ ID NO: 8 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPATTVLDHLSYFGINTGLCT

SEQ ID NO: 9 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPDTTVLDHLSYFGINTGLCT

SEQ ID NO: 10 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLECSDASNSIVPQTTVLDHLSYFGINTGLCT

SEQ ID NO: 11 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPNTSVLDHLSYFGINTGLCT

SEQ ID NO: 12 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLECSDASNSIVPATSVLDHLSYFGINTGLCT

SEQ ID NO: 13 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPDTSVLDHLSYFGINTGLCT

SEQ ID NO: 14 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPQTSVLDHLSYFGINTGLCT

SEQ ID NO: 15 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPCTSVLDHLSYFGINTGLCT

SEQ ID NO: 16 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPETSVLDHLSYFGINTGLCT

SEQ ID NO: 17 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPGTSVLDHLSYFGINTGLCT

SEQ ID NO: 18 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPHTSVLDHLSYFGINTGLCT

SEQ ID NO: 19 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPITSVLDHLSYFGINTGLCT

SEQ ID NO: 20 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPKTSVLDHLSYFGINTGLCT

SEQ ID NO: 21 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPLTSVLDHLSYFGINTGLCT

SEQ ID NO: 22 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPMTSVLDHLSYFGINTGLCT

SEQ ID NO: 23 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPPTSVLDHLSYFGINTGLCT

SEQ ID NO: 24 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPRTSVLDHLSYFGINTGLCT

SEQ ID NO: 25 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPSTSVLDHLSYFGINTGLCT

SEQ ID NO: 26 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPTTSVLDHLSYFGINTGLCT

SEQ ID NO: 27 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPVTSVLDHLSYFGINTGLCT

SEQ ID NO: 28 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPWTSVLDHLSYFGINTGLCT

SEQ ID NO: 29 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPYTSVLDHLSYFGINTGLCT

SEQ ID NO: 30 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDAVPHLPPAAFGFLHAGEEYWITDNSPETVQVCTSDLETSDCSNSIVPFTSVLDHLSYFGINTGLCT

SEQ ID NO: 31 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDDVPHLPPAAFGFLHAGEEYWITDNSPETVQVCTSDLETSDCSNSIVPFTSVLDHLSYFGINTGLCT

SEQ ID NO: 32 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDFVPHLPPAAFGFLHAGEEYWITDNSPETVQVCTSDLETSDCSNSIVPFTSVLDHLSYFGINTGLCT

SEQ ID NO: 33 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDNVPHLPPAAFGFLHAGEEYWITDNSPETVQVCTSDLETSDCSNSIVPFTSVLDHLSYFGINTGLCT

SEQ ID NO: 34 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDRVPHLPPAAFGFLHAGEEYWITDNSPETVQVCTSDLETSDCSNSIVPFTSVLDHLSYFGINTGLCT

SEQ ID NO: 35 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSALDHLSYFGINTGLCT

SEQ ID NO: 36 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSDLDHLSYFGINTGLCT

SEQ ID NO: 37 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSFLDHLSYFGINTGLCT

SEQ ID NO: 38 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSNLDHLSYFGINTGLCT

SEQ ID NO: 39 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSRLDHLSYFGINTGLCT

SEQ ID NO: 40 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPNTTVLDHLSYFGINTGLCT

SEQ ID NO: 41 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPNTSVLDHLSYFGINTGLCT

SEQ ID NO: 42 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLECSDASNSIVPATSVLDHLSYFGINTGLCT

SEQ ID NO: 43 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPDTSVLDHLSYFGINTGLCT

SEQ ID NO: 44 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPQTSVLDHLSYFGINTGLCT

SEQ ID NO: 45 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPCTSVLDHLSYFGINTGLCT

SEQ ID NO: 46 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPETSVLDHLSYFGINTGLCT

SEQ ID NO: 47 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPGTSVLDHLSYFGINTGLCT

SEQ ID NO: 48 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPHTSVLDHLSYFGINTGLCT

SEQ ID NO: 49 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPITSVLDHLSYFGINTGLCT

SEQ ID NO: 50 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPKTSVLDHLSYFGINTGLCT

SEQ ID NO: 51 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPLTSVLDHLSYFGINTGLCT

SEQ ID NO: 52 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPMTSVLDHLSYFGINTGLCT

SEQ ID NO: 53 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPPTSVLDHLSYFGINTGLCT

SEQ ID NO: 54 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPRTSVLDHLSYFGINTGLCT

SEQ ID NO: 55 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPSTSVLDHLSYFGINTGLCT

SEQ ID NO: 56 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPTTSVLDHLSYFGINTGLCT

SEQ ID NO: 57 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPVTSVLDHLSYFGINTGLCT

SEQ ID NO: 58 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPWTSVLDHLSYFGINTGLCT

SEQ ID NO: 59 - protein, 269 aa, Artificial Seqeunce
  Substituted RML
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPYTSVLDHLSYFGINTGLCT

SEQ ID NO: 60 - DNA, 21 bp, Artificial Seqeunce
  Synthetic primer
gactggttccaattgacaacg

SEQ ID NO: 61 - DNA, 21 bp, Artificial Seqeunce
  Synthetic primer
gcaaatggcattctgacatcc

SEQ ID NO: 62 - DNA, 36 bp, Artificial Seqeunce
  Synthetic primer
gtgtacaaacgatactgagacttccgactgctctaa

SEQ ID NO: 63 - DNA, 37 bp, Artificial Seqeunce
  Synthetic primer
aagtctcagtatcgtttgtacacacttgaacggtttc

SEQ ID NO: 64 - DNA, 39 bp, Artificial Seqeunce
  Synthetic primer
ttagagacttccaactgcactaacagtattgttccattt

SEQ ID NO: 65 - DNA, 32 bp, Artificial Seqeunce
  Synthetic primer
agtgcagttggaagtctctaaatcagatgtac

SEQ ID NO: 66 - DNA, 38 bp, Artificial Seqeunce
  Synthetic primer
gtattgttccaaacactactgttcttgatcatttgtct

SEQ ID NO: 67 - DNA, 35 bp, Artificial Seqeunce
  Synthetic primer
gaacagtagtgtttggaacaatactgttagagcag

SEQ ID NO: 68 - DNA, 38 bp, Artificial Seqeunce
  Synthetic primer
gtattgttccaaacacttcagttcttgatcatttgtct

SEQ ID NO: 69 - DNA, 36 bp, Artificial Seqeunce
  Synthetic primer
gaactgaagtgtttggaacaatactgttagagcagt

SEQ ID NO: 70 - DNA, 38 bp, Artificial Seqeunce
  Synthetic primer
gtattgttccagctacttcagttcttgatcatttgtct

SEQ ID NO: 71 - DNA, 35 bp, Artificial Seqeunce
  Synthetic primer
gaactgaagtagctggaacaatactgttagagcag

SEQ ID NO: 72 - DNA, 38 bp, Artificial Seqeunce
  Synthetic primer
gtattgttccagatacttcagttcttgatcatttgtct

SEQ ID NO: 73 - DNA, 35 bp, Artificial Seqeunce
  Synthetic primer
gaactgaagtatctggaacaatactgttagagcag

SEQ ID NO: 74 - DNA, 38 bp, Artificial Seqeunce
  Synthetic primer
gtattgttccacaaacttcagttcttgatcatttgtct

SEQ ID NO: 75 - DNA, 35 bp, Artificial Seqeunce
  Synthetic primer
gaactgaagtttgtggaacaatactgttagagcag

SEQ ID NO: 76 - DNA, 52 bp, Artificial Seqeunce
  Synthetic primer
cgactgctctaacagtattgttccatgtacttcagttcttgatcatttgtct

SEQ ID NO: 77 - DNA, 25 bp, Artificial Seqeunce
  Synthetic primer
tggaacaatactgttagagcagtcg

SEQ ID NO: 78 - DNA, 52 bp, Artificial Seqeunce
  Synthetic primer
cgactgctctaacagtattgttccagaaacttcagttcttgatcatttgtct

SEQ ID NO: 79 - DNA, 52 bp, Artificial Seqeunce
  Synthetic primer
cgactgctctaacagtattgttccaggtacttcagttcttgatcatttgtct

SEQ ID NO: 80 - DNA, 52 bp, Artificial Seqeunce
  Synthetic primer
cgactgctctaacagtattgttccacatacttcagttcttgatcatttgtct

SEQ ID NO: 81 - DNA, 52 bp, Artificial Seqeunce
  Synthetic primer
cgactgctctaacagtattgttccaattacttcagttcttgatcatttgtct

SEQ ID NO: 82 - DNA, 52 bp, Artificial Seqeunce
  Synthetic primer
cgactgctctaacagtattgttccaaagacttcagttcttgatcatttgtct

SEQ ID NO: 83 - DNA, 52 bp, Artificial Seqeunce
  Synthetic primer
cgactgctctaacagtattgttccattgacttcagttcttgatcatttgtct

SEQ ID NO: 84 - DNA, 52 bp, Artificial Seqeunce
  Synthetic primer
cgactgctctaacagtattgttccaatgacttcagttcttgatcatttgtct

SEQ ID NO: 85 - DNA, 52 bp, Artificial Seqeunce
  Synthetic primer
cgactgctctaacagtattgttccaccaacttcagttcttgatcatttgtct

SEQ ID NO: 86 - DNA, 52 bp, Artificial Seqeunce
  Synthetic primer
cgactgctctaacagtattgttccaagaacttcagttcttgatcatttgtct

SEQ ID NO: 87 - DNA, 52 bp, Artificial Seqeunce
  Synthetic primer
cgactgctctaacagtattgttccatctacttcagttcttgatcatttgtct

SEQ ID NO: 88 - DNA, 52 bp, Artificial Seqeunce
  Synthetic primer
cgactgctctaacagtattgttccaactacttcagttcttgatcatttgtct

SEQ ID NO: 89 - DNA, 52 bp, Artificial Seqeunce
  Synthetic primer
cgactgctctaacagtattgttccagttacttcagttcttgatcatttgtct

SEQ ID NO: 90 - DNA, 52 bp, Artificial Seqeunce
  Synthetic primer
cgactgctctaacagtattgttccatggacttcagttcttgatcatttgtct

SEQ ID NO: 91 - DNA, 52 bp, Artificial Seqeunce
  Synthetic primer
cgactgctctaacagtattgttccatacacttcagttcttgatcatttgtct

SEQ ID NO: 92 - DNA, 44 bp, Artificial Seqeunce
  Synthetic primer
cgtactgttaacgaaagagacgctgtaccacacttgcctccagc

SEQ ID NO: 93 - DNA, 29 bp, Artificial Seqeunce
  Synthetic primer
gtctctttcgttaacagtacgtctatatg

SEQ ID NO: 94 - DNA, 44 bp, Artificial Seqeunce
  Synthetic primer
cgtactgttaacgaaagagacgatgtaccacacttgcctccagc

SEQ ID NO: 95 - DNA, 44 bp, Artificial Seqeunce
  Synthetic primer
cgtactgttaacgaaagagactttgtaccacacttgcctccagc

SEQ ID NO: 96 - DNA, 44 bp, Artificial Seqeunce
  Synthetic primer
cgtactgttaacgaaagagacaacgtaccacacttgcctccagc

SEQ ID NO: 97 - DNA, 44 bp, Artificial Seqeunce
  Synthetic primer
cgtactgttaacgaaagagacagagtaccacacttgcctccagc

SEQ ID NO: 98 - DNA, 59 bp, Artificial Seqeunce
  Synthetic primer
ctaacagtattgttccatttacttcagctcttgatcatttgtcttattttggaattaac

SEQ ID NO: 99 - DNA, 29 bp, Artificial Seqeunce
  Synthetic primer
tgaagtaaatggaacaatactgttagagc

SEQ ID NO: 100 - DNA, 59 bp, Artificial Seqeunce
  Synthetic primer
ctaacagtattgttccatttacttcagatcttgatcatttgtcttattttggaattaac

SEQ ID NO: 101 - DNA, 59 bp, Artificial Seqeunce
  Synthetic primer
ctaacagtattgttccatttacttcatttcttgatcatttgtcttattttggaattaac

SEQ ID NO: 102 - DNA, 59 bp, Artificial Seqeunce
  Synthetic primer
ctaacagtattgttccatttacttcaaaccttgatcatttgtcttattttggaattaac

SEQ ID NO: 103 - DNA, 59 bp, Artificial Seqeunce
  Synthetic primer
ctaacagtattgttccatttacttcaagacttgatcatttgtcttattttggaattaac

(19) United States
(12) Patent Application Publication
(10) Pub. No.: US 2024/0175063 A1
(43) Pub. Date: May 30, 2024

(54) VARIANTS OF RHIZOMUCOR MIEHEI LIPASE AND USES THEREOF

(71) Applicant: WILMAR INTERNATIONAL LIMITED, Singpore (SG)

(72) Inventors: Junshi ZHENG, Singapore (SG); Oleg LATYPOV, Singapore (SG)

(21) Appl. No.: 18/552,468

(22) PCT Filed: Mar. 30, 2022
(86) PCT No.: PCT/SG2022/050180

§ 371 Date: Sep. 26, 2023

(30) Foreign Application Priority Data
Mar. 30, 2021 (SG) 10202103274U

(51) Int. Cl. C12P 7/6418; C12N 9/20; C12N 11/08

(52) U.S. Cl. CPC C12P 7/6418; C12N 9/20; C12N 11/08; C12Y 301/01003

(57) ABSTRACT

Disclosed herein is a lipase variant comprising a first substituent of a lipase sequence of SEQ ID No. 2, wherein a position of the first substituent in SEQ ID No. 2 is selected from the group consisting of position 251, position 204, position 254, position 237, and position 243. The lipase variant may be used in a method to react with a first ester and a reactant, wherein the reactant is selected from the group consisting of water, an acid and a second ester; and forming a reaction product between the first ester and the reactant with the aid of the lipase variant under suitable reaction conditions.

The present application claims priority to Singapore patent application number 10202103274U titled “Variants of Rhizomucor miehei lipase and uses thereof” filed on 30 Mar. 2021 which is incorporated by reference herein in its entirety.

### BACKGROUND

Triacylglycerol lipases belong to the class of hydrolases, which cleave the ester bond of carboxylic esters (E.C. 3.1.1.). The specific property of lipases is their ability to efficiently cleave the ester bond between glycerol and fatty acids of lipids—different types of natural fats and oils. Currently, lipases are one of the most widely used class of enzymes with applications found in a wide range of industries including the detergent, pharmaceutical, food and biofuel industries.

Rhizomucor miehei lipase (RML) (mature peptide shown as SEQ ID No. 2) is one of the more commonly used lipases in industrial processes. One of its applications is in the production of human milk fat substitutes where it can perform transesterification or acidolysis. For different processes, the requirements for properties such as thermostability, chemical resistance, and pH for the optimal activity, as well as substrate specificity, of lipases differs. To suit the applications of RML, engineering efforts have been made to improve its thermostability (Zhang et al., 2012) and enantioselectivity (Holmquist et al., 1993). However, regardless of application, specific activity remains as one of the most important properties of lipases since higher specific activity of enzyme allows savings in both enzyme and time. Thus, we have engineered variants of RML with improved activity.

Several ways are currently known for increasing specific activity of lipases. The most popular ways include manipulations of the substrate-binding pocket (Lafaquière et al., 2009), the lid domain (Khan et al., 2017) and the surface loops (Yedavalli et al., 2013). These manipulations can be achieved through random mutagenesis or site-directed mutagenesis. Previously, site saturation mutagenesis had been successfully employed to identify RML variants with higher specific activity and greater stability for use in detergents (Balumuri et al., 2015).

### SUMMARY

In a first aspect of the invention, there is provided a lipase variant comprising a first substituent of a lipase sequence of SEQ ID No. 2, wherein a position of the first substituent in SEQ ID No. 2 is selected from the group consisting of position 251, position 204, position 254, position 237, and position 243. Preferably, the first substituent is less hydrophobic than phenylalanine.

Preferably, the position of the first substituent is at position 251 and the first substituent is selected from the group consisting of asparagine, alanine, cysteine, aspartic acid, glutamic acid, glycine, histidine, isoleucine, lysine, leucine, methionine, proline, glutamine, arginine, serine, threonine, valine, tryptophan and tyrosine. Advantageously, the phenylalanine at position 251 of the wild type may be substituted with any other natural amino acid.

Preferably, the position of the first substituent is at position 204 and the first substituent is selected from the group consisting of more hydrophobic, less hydrophobic, polar and uncharged, negatively charged and positively charged, wherein the charge is determined with respect to the first substituent at a pH of 7. Preferably, the position of the first substituent is at position 204 and the first substituent is selected from the group consisting of phenylalanine, leucine, methionine, tyrosine, tryptophan, alanine, valine, glycine, methionine, proline, serine, threonine, asparagine, glutamine, aspartic acid, glutamic acid, arginine, histidine, and lysine. More preferably, the first substituent is selected from the group consisting of alanine, aspartic acid, phenylalanine, asparagine, and arginine Advantageously, the isoleucine at position 254 of the wild type may be replaced by any natural amino acid.

Preferably, the position of the first substituent is at position 254 and the first substituent is selected from the group consisting of less hydrophobic, polar, and negatively charged, wherein the charge is determined with respect to the first substituent at a pH of 7. Preferably, the position of the first substituent is at position 254 and the first substituent is selected from the group consisting of methionine, tyrosine, cysteine, alanine, glycine, serine, threonine, asparagine, glutamine, aspartic acid, and glutamic acid. More preferably, the first substituent is selected from the group consisting of alanine, aspartic acid, and asparagine.

Preferably, the lipase variant comprises a second substituent of the lipase sequence of SEQ ID No.2, wherein the position of the second substituent in SEQ ID No. 2 is position 253, wherein the first substituent is preferably at position 251. Preferably, the second substituent is an amino acid with a polar uncharged side chain, for example threonine, asparagine, and glutamine, preferably threonine. More preferably, the first substituent is at position 251 and selected from the group consisting of asparagine, alanine, aspartic acid, and glutamine.

Preferably, the lipase variant comprises a third substituent of the lipase sequence of SEQ ID No.2, wherein the position of the third substituent in SEQ ID No. 2 is position 156, wherein the first substituent is preferably at position 251. Preferably, the third substituent may be glycine. The D156X (preferably D156G) substitution may be paired with the F251X mutation (where X is any other amino acid) alone or in combination with F251X and S253X (preferably S253T) mutations.

Preferably, the first substituent is at position 251 and the lipase variant comprises a fourth substituent and a fifth substituent, wherein the fourth substituent and the fifth substituent is either at positions 237 and 239 respectively or positions 243 and 245 respectively. The fourth and fifth substituent may each independently be an amino acid with a polar uncharged side chain. For example, asparagine, threonine, serine, and glutamine as appropriate. In an example, the fourth substituent is asparagine, and the fifth substituent is threonine. Advantageously, the double substitution of asparagine and threonine (with an interposing amino acid, in other words 2 positions away) provide lipase variants with improved activity compared to the RML wild type.

Preferably, the lipase variant comprises a sequence selected from the group consisting of SEQ ID No. 3, SEQ ID No. 4, SEQ ID No. 5, SEQ ID No. 6, SEQ ID No. 7, SEQ ID No. 8, SEQ ID No. 9. SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19. SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 56, SEQ ID No. 57, SEQ ID No. 58, and SEQ ID No. 59.

Preferably, the lipase variant comprises a sixth substituent of a lipase sequence of SEQ ID No. 2, wherein the first substituent and the sixth substituent are at or proximal to a surface loop region of the lipase sequence, wherein the first substituent is asparagine and the sixth substituent is threonine, the sixth substituent being two amino acid positions away from the first substituent. More preferably, the first substituent and sixth substituent are upstream of position 257.

Preferably, the first substituent and sixth substituent is selected from the group consisting of S237N and L239T, D243N and S245T, and F251N and S253T.

For example, the lipase variant may comprise any one of SEQ ID No. 3, SEQ ID No. 4, SEQ ID No. 5, SEQ ID No. 6, and SEQ ID No. 7.

In various embodiments, the lipase variant has at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the sequence of SEQ ID NO: 2.

In all aspects of the invention, the lipase variant comprises, or consists essentially of, or consists of, the substituent/s or SEQ ID No as described herein in the specification. For example, the lipase variant may comprise, or consists essentially of, or consists of, the first substituent of SEQ ID No. 2, and optionally additional substituents. For example, the lipase variant may comprise, or consists essentially of, or consists of, any one of the SEQ ID No. recited above.

The charge of the amino acid side chain described herein is determined in water at pH 7 at 25° C. The relative hydrophobicity of the amino acid side chain described herein is determined in water at pH 7, an example of a listing of the hydrophobicity of the natural amino acids is described in Monera et al. J. Pept. Sci., 1995, 1, 319-329.

In another aspect of the invention, there is provided a method comprising providing the lipase variant as disclosed herein or in the first aspect, a first ester and a reactant, wherein the reactant is selected from the group consisting of water, an acid and a second ester; and forming a reaction product between the first ester and the reactant with the aid of the lipase variant under suitable reaction conditions. The lipase variant may be used in different reactions including the hydrolysis of the first ester, and transesterification reactions with an acid or a different (i.e. a second) ester. More than one additional acid or ester as the reactant may be used. The transesterification reactions may be done in the absence of water or with minimal water present to avoid competing hydrolysis reactions. The lipase may catalyse the reaction between the reactants or via an intermediate which is not isolated.

The term “ester” as used herein includes fatty acid esters of glycerol like monoglyceride, diglyceride, triglyceride, and monoesters formed from short, medium, and long chain fatty acids and short chain alcohols like methanol, ethanol, propanol, and butanol including all structural isomers.

Preferably, the first ester is a fatty acid ester preferably a triacylglyceride. More preferably, the fatty acid ester or triacylglyceride is selected from the group consisting of olive oil, castor oil, sunflower oil, high oleic sunflower oil, rapeseed oil, high oleic rapeseed oil, palm oil, palm fraction enriched in tripalmitin, shea butter, shea olein, canola oil, glycerol trioctanoate, tributyrin, methyl octanoate, methyl decanoate, methyl dodecanoate, and vinyl laurate.

In an embodiment, the reactant is a medium chain fatty acid or ester. For example, the reactant is selected from the group consisting of octanoate (C8) ester, decanoate (C10) ester, dodecanoate (C12) ester, and mixtures thereof. The corresponding medium chain fatty acids may be used as well. Advantageously, this provides medium- and long-chin triacylglycerols (MLCT). For example, the reaction product may be a triacylglyceride with medium chain fatty acids at the 1,3-position of the triacylglyceride and a long chain fatty acid at the 2-position of the triacylglyceride. In other words, the reaction product has a formula of R¹OCH₂CH(OR²)CH₂OR¹ wherein R¹ is a medium chain fatty acid and R² is a long chain fatty acid.

In an embodiment, the reactant is a long chain fatty acid or ester. Examples include palmitic acid, stearic acid, oleic acid, linoleic acid and their corresponding esters.

In an embodiment, the first ester is palm oil and the reactant is oleic acid, oleic ester, linoleic acid or linoleic ester. Preferably, the first ester is palm fraction enriched in tripalmitin (glycerol tripalmitate). Palm fraction refers to the isolation of a fraction of palm oil.

In an embodiment, the fatty acid ester or triacylglyceride is selected from the group consisting of olive oil, high oleic sunflower oil, and high oleic rapeseed oil, and the reactant is stearic acid or stearic ester. The reaction product formed would be 1,3-distearoyl-2-oleoyl glycerol (SOS).

Preferably, the variant is SEQ ID No. 5. Preferably, water is provided as an aqueous buffer. Preferably, the variant is immobilized on a resin.

Preferably, the first ester and the reactant are in a liquid phase or a solution phase under the suitable reaction conditions. In other words, the first ester and reactant are miscible liquids or dissolved in solution, for example a solvent is used to dissolve the first ester and reactant, or either the first ester or reactant acts as a solvent to dissolve the other.

Preferably, the reactant is the second ester, and the reaction product is a transesterification product of the first ester and the second ester. For example, one of the esters may be palm oil or olive oil and the other ester may be at least one of a C8, C10, or C12 methyl ester, or any combinations thereof. In particular, it may be a mixture of C8, C10, and C12 methyl esters.

It will be appreciated that the MLCT product and SOS product (and other products formed by the methods herein) may be the major product formed but is unlikely to be the only product formed.

In another aspect of the invention, there is provided a product obtained from the methods described above.

### DETAILED DESCRIPTION OF EMBODIMENTS OF THE INVENTION

In the Figures:

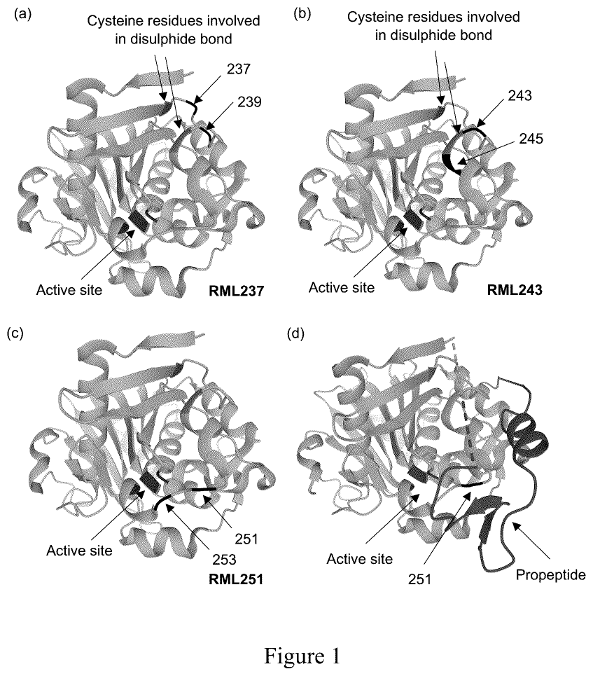

FIG. 1 shows the ribbon diagram of RML (PDB ID-3TGL; Brzozowski et al., 1993; PDB ID-6QPP; Moroz et al., 2019; Sehnal et al., 2018). Arrows indicate the positions of the amino acids 237 and 239, 243 and 245, and 251 and 253 which are mutated for (a) RML 237, (b) RML 243 (b) and (c) RML 251 respectively. Panel (d) of FIG. 1 shows the position of amino acid 251 relative to the non-covalently bound propeptide.

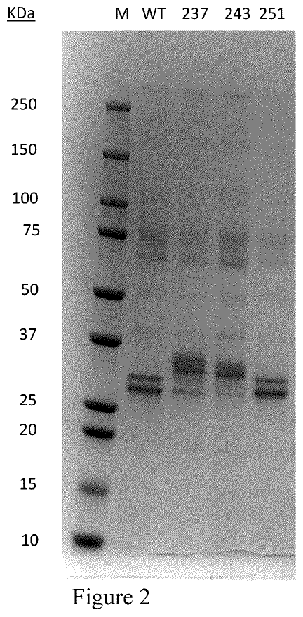

FIG. 2 shows the SDS-PAGE analysis of secreted protein in P. Pichia with heterologous expression of RML. The gel was stained with Coomassie blue for visualization. M, marker; WT, wild-type.

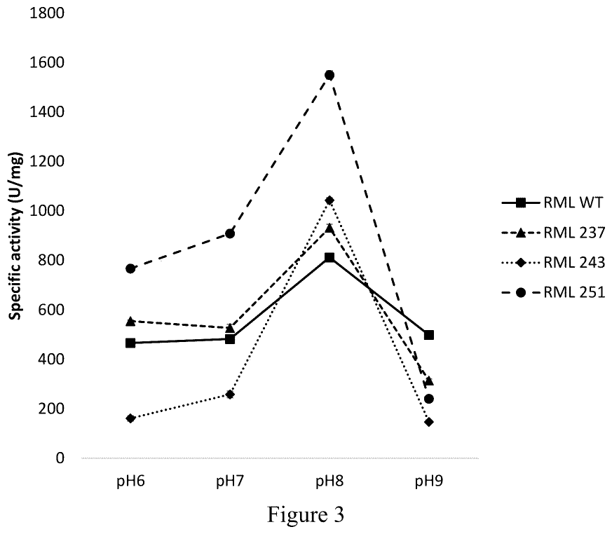

FIG. 3 shows the specific activity of RML variants at various pH. Olive oil was used as substrate for the lipase activity assays at a pH of between 6 to 9. The values are expressed as mean±SD (n=3).

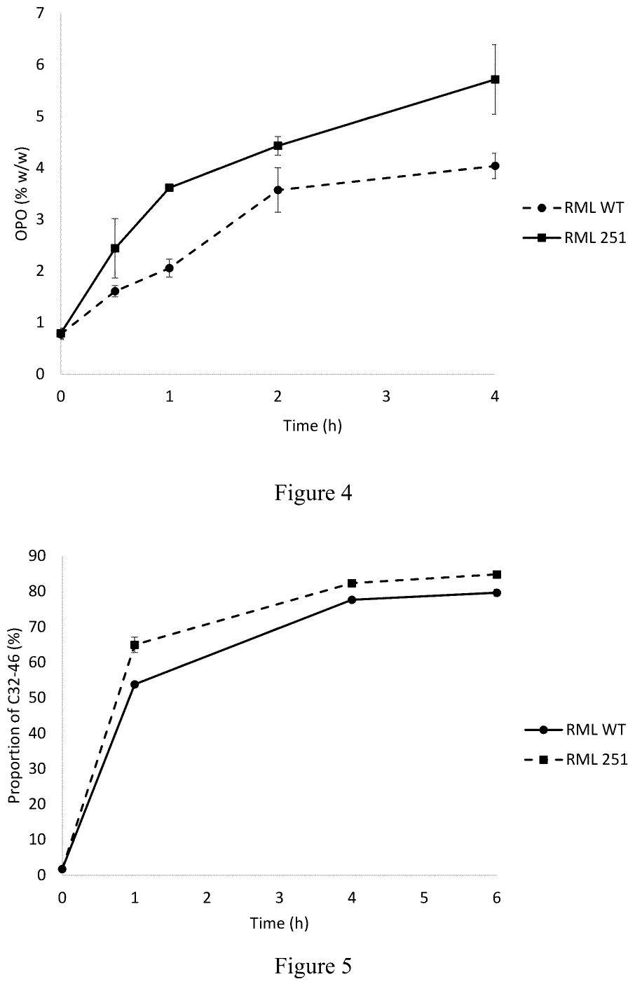

FIG. 4 shows 1,3-Dioleoyl-2-palmitoylglycerol (OPO) production by acidolysis using RML. OPO content (% w/w) in reaction mixture was measured during the first four hours of incubation with immobilized RML WT and RML 251. The values are expressed as mean±SD (n=3).

FIG. 5 shows medium and long chain triacylglycerol (MLCT) production by transesterification using RML and RML251 (SEQ ID No. 5). C32 to C46 content (% w/w) in reaction mixture was measured for the first six hours of incubation with immobilized RML WT and RML 251. The values are expressed as mean±SD (n=3).

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The following detailed description is merely exemplary in nature and is not intended to limit the invention or the application and uses of the invention. Furthermore, there is no intention to be bound by any theory presented in the preceding background of the invention or the following detailed description.

Other technical advantages may become readily apparent to one of ordinary skill in the art after review of the following figures and description.

Various amino acids are described herein by its full name, and conventional 1-letter and 3-letter abbreviations as is known in the art. The substitution of an amino acid residue in a peptide is describe by the conventional notation, for example F251N indicates that the 251^{th} amino acid residue (or position) of phenylalanine (F) is substituted by asparagine (N).

As used herein, the articles “a”, “an” and “the” as used with regard to a feature or element include a reference to one or more of the features or elements. As used herein, the term “and/or” includes any and all combinations of one or more of the associated listed items. As used herein, the terms “first,” “second,” and “third,” etc. are used merely as labels, and are not intended to impose numerical requirements on their objects. For example, the lipase variant may comprise the first substituent, and the third substituent without having the second substituent. In another example, the lipase variant may comprise the first substituent, and the sixth substituent without having the second substituent, third substituent, fourth substituent, and fifth substituent. Other examples of different substituents are also possible. Thus, the terms “first,” “second,” and “third,” etc do not impose any numerical requirement.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

The terms “sequence similarity”, “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity is evaluated using any of the variety of sequence comparison algorithms and programs known in the art. Such algorithms and programs include, but are by no means limited to, TBLASTN, BLASTP, FASTA, TFASTA, CLUSTAL W, FASTDB.

The surface loop (positions 236-243 and 250 to 253 of SEQ ID No. 2) in the vicinity of the substrate binding pocket is modified by introduction of a N-glycosylation site (asparagine) to generate variants with increased hydrolytic activity between 34% and 115%. Additionally, variants were made with substituted amino acids at a region that could potentially interact with the propeptide. Structural studies of RML have suggested that, despite cleavage of the propeptide after protein maturation, it can remain in contact with the mature protein at the lid region to inhibit its activity (Moroz et al., 2019). Modifications of the amino acid at regions of contact could potentially dislodge the non-covalently attached propeptide. It may be possible that the sidechain of the substituted amino acids in RML may disrupt the interactions with the propeptide leading to improved activity of the modified RML. Through this method, improved hydrolytic activity towards olive oil by up to 153% has been obtained. The variants also demonstrated varying degrees of improvement in hydrolysis towards a wide range of substrates including castor oil, glycerol trioctanoate, tributyrin, C12 methyl ester, C8/C10 methyl esters and vinyl laurate.

### EXAMPLES

Site directed mutagenesis was performed to introduce mutations at amino acids in the vicinity of the substrate binding pocket (at or proximal to the surface loop region), in particular a pair of mutations comprising asparagine and threonine with an unmodified amino acid in between. The asparagine modification provides an N-glycosylation site and the threonine may promote the glycosylation. The following substitutions S237N+L239T, D243N+S245T and F251N+S253T were introduced and identified as variants RML237, RML243 and RML251, respectively (FIG. 1). Compared to WT RML, the hydrolytic activities of RML237, RML243 and RML251 towards olive oil improved by 34%, 63% and 168%, respectively (results shown in Table 1). When the substrate was changed to castor oil, an improvement of 58% was also seen in RML237 and an increase of 253% was achieved by RML251 (Table 1). The hydrolysis of C8-C10 methyl esters was slightly improved by 20% with RML 237 and further increased by 96% with RML 251 but RML 243 showed decreased hydrolytic activity compared to the wild type RML.

TABLE 1
Relative hydrolytic activity of RML variants on olive oil and
castor oil. The values are expressed as mean of triplicates.

| | Relative specific activity (%) | | |
|---|---|---|---|
| | Olive oil | Castor Oil | C8/C10 methyl esters |
| RML WT | 100 | 100 | 100 |
| RML 237 | 134 | 158 | 120 |
| RML 243 | 163 | 84 | 12 |
| RML 251 | 268 | 353 | 196 |
| RML 237 + 251 | 224 | — | — |
| RML 243 + 251 | 118 | — | — |

In terms of optimal temperature, a shift was observed amongst the variants as well. Instead of having an optimal temperature for hydrolysis at 55° C. like for WT, RML237 and RML 251 achieved its highest activity at 50° C., while RML 243 has lowest optimal temperature of 40° C. These variants which require lower temperatures for optimal activity could help minimise the need for heating and thus save on energy costs. The optimal pH for activity remained at 8 for the variants and the activity at various pH are shown in FIG. 4.

Variant with combined mutations RML 237+251 demonstrated an increase of 124% in specific activity compared to the wild type RML (WT). RML 243+251 was 18% more active than WT (Table 1).

RML 251 was immobilized and used for acidolysis to produce 1,3-Dioleoyl-2-palmitoylglycerol (OPO), the amount of OPO accumulated was 51.8% more than RML WT after the first 30 min of reaction and 75.9% and 41.6% higher than RML at the end of 1 h and 4 h respectively (FIG. 4). The immobilized RML 251 was also used for transesterification to produce medium and long chain triacylglycerol (MLCT). At the end of 1 h reaction, the proportion of C32-C46 accumulated was 20.7% higher for RML 251 than WT and continued to be slightly higher than WT at around 6% after 4 h and 6 h (FIG. 5).

By replacing the amino acid at position 251 to other less hydrophobic amino acid or hydrophilic amino acids, the variants still had activities that are comparable to RML 251. For variants with double mutation of F251A/D/Q+S253T, their specific activities were 98 to 128% higher than WT for olive oil hydrolysis (Table 2). RML F251A+S253T had the same optimal temperature as RML 251 of 50° C. while the other two variants, RML F251D+S253T and F251Q+S253T, had slightly lower optimal temperature of 45° C. (Table 3).

When the S253T mutation of RML 251 was removed, we noticed that the resulting RML F251N variant had comparable activity to RML 251 and had optimal temperature for olive oil hydrolysis at 50° C. as well. Thus, for further analysis site saturation mutagenesis was performed at amino acid position 251 only.

All variants generated by saturated mutagenesis at position 251, except RML F251W and RML F251Y, exhibited significant improvements in specific activity of between 109 to 281% compared to WT using olive oil as the substrate (Table 2). However, the F251W and F251Y were found to have slightly higher activity than the WT at 60° C. and thus may still be useful in certain processes. By changing the substrate for hydrolysis to castor oil, glycerol trioctanoate, tributyrin, C12 methyl ester, C8/C10 methyl esters and vinyl laurate, specific activities of these F251 variants were between 109 to 922% higher than WT. RML F251Y, which demonstrated a small improvement of 22% at olive oil hydrolysis also had a more modest improvement of between 31-77% for the seven other substrates. RML F251W, which had similar activity as WT at hydrolysing olive oil, also had comparable activity at the hydrolysis of other substrates studied. (Table 2). Among these variants, eight maintained the optimal temperature of 55° C. like in WT, while the remaining eleven variants had the same optimal temperature of 50° C. as RML 251.

TABLE 2
Relative hydrolytic activity of RML variants with mutations at amino acid position 251. Olive oil,
castor oil, glycerol trioctanoate, tributyrin, C12 methyl ester, C10 methyl ester, C8 methyl ester
and vinyl laurate were used as substrates. The values are expressed as mean of triplicates.

| | Relative activity (%) | | | | | | | |
|---|---|---|---|---|---|---|---|---|
| | | | | | C12 | C10 | C8 | |
| | Olive | Castor | Glycerol | | methyl | methyl | methyl | Vinyl |
| | oil | oil | trioctanoate | Tributyrin | ester | ester | ester | laurate |
| RML WT | 100 | 100 | 100 | 100 | 100 | 100 | 100 | 100 |
| RML 251 | 268 | 353 | 249 | 316 | 286 | 297 | 258 | 342 |
| (F251N + S253T) | | | | | | | | |
| RML | 198 | | | | | | | |
| F251A + S253T | | | | | | | | |
| RML | 202 | | | | | | | |
| F251D + S253T | | | | | | | | |
| RML | 228 | | | | | | | |
| F251Q + S253T | | | | | | | | |
| RML F251N | 248 | 284 | 217 | 284 | 268 | 289 | 377 | 277 |
| RML F251A | 219 | 282 | 245 | 510 | 325 | 355 | 463 | 275 |
| RML F251C | 254 | 335 | 274 | 255 | 442 | 475 | 591 | 303 |
| RML F251D | 245 | 286 | 260 | 527 | 325 | 345 | 377 | 303 |
| RML F251E | 272 | 277 | 263 | 274 | 430 | 425 | 520 | 301 |
| RML F251G | 249 | 332 | 278 | 270 | 470 | 472 | 526 | 322 |
| RML F251H | 209 | 253 | 232 | 215 | 325 | 299 | 389 | 262 |
| RML F251I | 232 | 306 | 270 | 263 | 459 | 450 | 551 | 318 |
| RML F251K | 237 | 327 | 241 | 275 | 515 | 491 | 414 | 330 |
| RML F251L | 263 | 394 | 295 | 279 | 456 | 448 | 545 | 327 |
| RML F251M | 281 | 302 | 209 | 209 | 327 | 286 | 333 | 258 |
| RML F251P | 210 | 370 | 321 | 319 | 592 | 719 | 1022 | 329 |
| RML F251Q | 216 | 282 | 248 | 482 | 268 | 302 | 381 | 281 |
| RML F251R | 249 | 311 | 249 | 249 | 482 | 516 | 569 | 311 |
| RML F251S | 271 | 321 | 224 | 224 | 445 | 425 | 355 | 306 |
| RML F251T | 247 | 304 | 262 | 262 | 473 | 538 | 613 | 303 |
| RML F251V | 254 | 340 | 283 | 283 | 473 | 550 | 638 | 332 |
| RML F251W | 96 | 103 | 113 | 113 | 114 | 91 | 97 | 118 |
| RML F251Y | 122 | 162 | 141 | 141 | 168 | 139 | 181 | 157 |

The optimum temperature for hydrolysis of olive of the RML WT and variants are summarized in Table 3.

TABLE 3
Optimum temperature for olive oil
hydrolysis for RML WT and variants.

| | Optimum temperature (° C.) |
|---|---|
| RML WT | 55 |
| RML 237 | 50 |
| RML 243 | 40 |
| RML 251 | 50 |
| RML F251A + S253T | 50 |
| RML F251D + S253T | 45 |
| RML F251Q + S253T | 45 |
| RML F251N | 50 |
| RML F251A | 50 |
| RML F251C | 50 |
| RML F251D | 50 |
| RML F251E | 50 |
| RML F251G | 50 |
| RML F251H | 55 |
| RML F251I | 55 |
| RML F251K | 55 |
| RML F251L | 55 |
| RML F251M | 50 |
| RML F251P | 55 |
| RML F251Q | 50 |
| RML F251R | 55 |
| RML F251S | 50 |
| RML F251T | 50 |
| RML F251V | 50 |
| RML F251W | 55 |
| RML F251Y | 55 |

All the mutation sites on RML237, RML243 and RML251 were introduced upstream of one of the residues of the catalytic triad, the His residue at position 257. For RML237, the mutations were located near a bend which is held in place by a disulphide bond (FIG. 1a), and for RML243, the mutations were straddling the cysteine residue involved in the disulphide bond itself (FIG. 1b). Hence, in these two variants, there may be potential disruptions to the disulphide bond formation, which may increase the flexibility of the strand involved in the formation of the substrate binding pocket. In RML251 (F251N+S253T), the two sites of mutations were located at a loop region in the vicinity of H257 and could thus potentially contribute to modifications in the substrate binding pocket as well (FIG. 1c). Furthermore, the propeptide, which inhibits RML activity, could interact with the amino acid at position 251, therefore replacing the residue with a hydrophilic residue or a hydrophobic residue with less bulky side chain could have contributed to reduced interactions between the propeptide and mature protein and promoted higher activity. Together with the observed differences in substrate specificity and optimal temperature for activity, these variants seemed to have achieved higher hydrolytic activity through modifications of the structure leading to increased flexibility of substrate binding pocket and also through reduced interactions with the inhibiting propeptide. It is possible that the substitution of one or more of the amino acids at other positions that may come into contact with the propeptide may also reduce the interaction of the propeptide with the lipase. For example, modification of I204 to other amino acids that are more hydrophobic, less hydrophobic, polar, negatively charged and positively charged, represented by Phe, Ala, Asn, Asp, Arg respectively, all showed improvements in hydrolytic activity of between 27-89% (Table 4). Modification of V254 to less hydrophobic, polar and negatively charged residues, represented by Ala, Asn and Asp, respectively, could also improve the hydrolytic activity by between 11-90% (Table 4).

TABLE 4
Relative hydrolytic activity of RML I204 and RML
V254 variants using olive oil as substrate

| | Relative activity (%) |
|---|---|
| RML WT | 100 |
| RML I204A | 147 |
| RML I204D | 189 |
| RML I204F | 127 |
| RML I204N | 169 |
| RML I204R | 142 |
| RML V254A | 111 |
| RML V254D | 168 |
| RML V254F | 76 |
| RML V254N | 190 |
| RML V254R | 73 |

When an additional mutation of Asp to Gly was introduced at amino acid position 156 on the variants with mutations at 251, further improvements in activity were observed. RML F251W and RML F251Y mutants, which alone only showed slight improvement, became 149-717% more active than WT with this additional D156G mutation (Table 5). For all other variants with the D156G mutation, the most significant improvements were observed for the hydrolysis of

C8-C12 methyl esters and castor oil (Table 5). The D156G mutation might have contributed to the higher activity due to modifications in the substrate binding pocket since Asp 156 is located on a helix structure where a Ser residue of the catalytic triad is also found.

TABLE 5
Relative hydrolytic activity of RML variants with D156G mutation on top of mutations
at amino acid position 251. Olive oil, castor oil, glycerol trioctanoate, tributyrin,
C12 methyl ester, C10 methyl ester, C8 methyl ester and vinyl laurate were used
as substrates. The values are expressed as mean of triplicates.

| | Relative activity (%) | | | | | | | |
|---|---|---|---|---|---|---|---|---|
| | | | | | C12 | C10 | C8 | |
| | Olive | Castor | Glycerol | | methyl | methyl | methyl | Vinyl |
| | oil | oil | trioctanoate | Tributyrin | ester | ester | ester | laurate |
| RML WT | 100 | 100 | 100 | 100 | 100 | 100 | 100 | 100 |
| RML 251 + D156G | 354 | 453 | 336 | 310 | 512 | 365 | 529 | 350 |
| RML F251N + D156G | 331 | 362 | 314 | 278 | 525 | 442 | 680 | 309 |
| RML F251A + D156G | 259 | 279 | 288 | 265 | 531 | 527 | 734 | 265 |
| RML F251C + D156G | 284 | 476 | 327 | 327 | 628 | 711 | 1057 | 333 |
| RML F251D + D156G | 247 | 433 | 301 | 290 | 536 | 575 | 750 | 287 |
| RML F251E + D156G | 224 | 357 | 283 | 266 | 509 | 510 | 798 | 256 |
| RML F251G + D156G | 234 | 321 | 296 | 270 | 534 | 537 | 773 | 280 |
| RML F251H + D156G | 220 | 419 | 323 | 324 | 609 | 639 | 891 | 320 |
| RML F251I + D156G | 213 | 359 | 296 | 284 | 562 | 580 | 854 | 289 |
| RML F251K + D156G | 219 | 384 | 311 | 251 | 587 | 566 | 794 | 302 |
| RML F251L + D156G | 243 | 362 | 310 | 294 | 568 | 530 | 789 | 311 |
| RML F251M + D156G | 257 | 338 | 298 | 266 | 492 | 437 | 699 | 293 |
| RML F251Q + D156G | 223 | 384 | 311 | 304 | 595 | 554 | 800 | 297 |
| RML F251R + D156G | 215 | 350 | 311 | 286 | 574 | 551 | 822 | 304 |
| RML F251S + D156G | 219 | 338 | 273 | 209 | 462 | 443 | 528 | 261 |
| RML F251T + D156G | 280 | 310 | 261 | 214 | 397 | 387 | 480 | 220 |
| RML F251V + D156G | 250 | 362 | 317 | 286 | 555 | 898 | 852 | 302 |
| RML F251W + D156G | 249 | 402 | 252 | 249 | 402 | 751 | 600 | 269 |
| RML F251Y + D156G | 281 | 433 | 254 | 255 | 473 | 817 | 683 | 300 |

### RML Mutagenesis and Expression

To generate RML variants, site directed mutagenesis was used to introduce substitution into wild-type (WT) RML. The codon optimized sequence (SEQ ID No. 1) for WT RML was synthesized based on its amino acid sequence (UniProtKB—P19515) for optimal expression in Pichia pastoris. The propeptide region had been modified for improved expression in Pichia. Mutated RML were amplified from pAO815W-WT RML construct using AOX promoter and terminator primers paired with primers designed to substitute the amino acid sequence at the targeted sites (Table 6) and cloned into the pAO815W vector at the HindIII and EcoRI sites. The pAO815W vector was modified from pAO815 with the removal of the HindIII site within the 5′-AOX promoter region and its reintroduction downstream of the 5′-AOX promoter. The resulting pAO815W-RML variant constructs were transformed into P. pastoris GS115 by electroporation as described previously (Wu et al., 2004). For the expression of RML variants, cells from a single colony were cultured overnight at 30° C. in shake flasks containing buffered glycerol-complex medium (BMGY; 1% yeast extract, 2% peptone, 100 mM potassium phosphate, pH 6.0, 1.34% YNB, 4×10⁻⁵% biotin and 1% glycerol). On the following day, the cells were pelleted and resuspended in equal volumes of buffered methanol-complex medium (BMMY; 1% yeast extract, 2% peptone, 100 mM potassium phosphate, pH 6.0, 1.34% YNB, 4×10⁻⁵% biotin and 0.5% methanol) with OD₆₀₀ normalized to that of the slowest growing culture, which is between 400-450. For the next three days, methanol (0.5% v/v) was supplemented to the cultures twice daily. On the 6^{th} day, culture media containing the secreted RML were collected and clarified by centrifugation. Cultural liquid was analyzed using SDS-PAGE (FIG. 2) to confirm for the presence of RML and its concentration was determined from Bradford assays using bovine serum albumin as a reference.

### Lipase Activity Assay

Determination of specific activity for the different RML variants was performed using olive oil, castor oil, glycerol trioctanoate, tributyrin, C12 methyl ester, C8/C10 methyl esters and vinyl laurate emulsion as substrate. The emulsion was prepared by homogenizing the oil with 4% (w/v) poly vinyl alcohol 30 000 solution in a ratio of 1:3 using a knife homogenizer and used immediately. For the assay, 2 ml of the oil emulsion, 1.5 ml of dH₂0 and 1 ml of 0.2 M Tris-HCl buffer, pH8.0 were first added to a 100 ml flat bottom flask and incubated in a 40° C. water-bath with shaking at 150 rpm. For the determination of optimal pH for activity, the buffer was replaced with 0.2 M Bis-Tris at pH 6 and 7, and 0.2 M Glycine-Sodium hydroxide buffer at pH 9. After 5 min, 500 μl of the enzyme solution (10 μg/ml) was added to the reaction mixture and incubated for a further 15 mins. 5 ml of absolute ethanol was then added to terminate the reaction. The amount of free fatty acid released during the reaction was determined through titration with 50 mM NaOH using phenolphthalein as an indicator. The specific activity (U/mg) of each RML variant was calculated using the following equation:

$$Specific activity ( U / mg ) = \frac{( v - {v}_{0} ) * C}{t * A}$$

v: Volume NaOH required for neutralization of reaction mix (ml)
v₀: Volume of NaOH required for neutralization of blank (ml)
C: Concentration of NaOH (μmol/ml)
t: Reaction time (min)
A: Amount of RML added (mg)

1 U corresponds to 1 μmol of free fatty acid released from the hydrolysis of olive oil in 1 min.

### OPO and MLCT Assay

To determine efficiency of RML251 for OPO and MLCT production, the lipase (RML251) was first immobilized on a hydrophobic resin. Non-limiting examples of a hydrophobic resin that may be used include: Lifetech™ ECR8806M, Lewatit® VP OC 1600 and AmberLite™ XAD™ 7HP Polymeric Adsorbent. Other resins such as ion exchange resin or mixed-mode resin may be used as well. A homogenized mixture containing palm oil and oleic acid was used for the OPO assay, and was melted and aliquoted into 2 ml tubes as the substrate and immobilized RML was added at 6% (w/v) dosage. The reaction was performed on a thermomixer at 60° C. with shaking at 1500 rpm. At the required time point, the samples were centrifuged at 14 000 rpm for one min to separate the immobilized RML. The top fraction was stored at −20° C. until analysis by LC-MS. The transesterification reaction between palm oil and oleic acid takes place in the absence of water or with minimal water present. A homogenized mixture containing high olein sunflower oil and medium chain triacylglycerol (with a mixture of C8 and C10 fatty acids, Wilfarester MCT) was used for the MLCT assay with the same procedure as for the OPO assay. GC-MS analysis was performed to determine the C32-C46 content.

In addition, it may be possible that the lipase variants described herein may be used to perform a transesterification reaction of two or more esters to produce the new ester product (transesterification product).

### REFERENCES

Balumuri, P., Saikia, R., Svendsen. A., Baunsgaard, L. 2015. Lipase variants and polynucleotides encoding same. WO 2015/181119 A2
Brzozowski, A M., Derewenda, Z S., Dodson, E J., Dodson, G G., Turkenburg, J. P. 1992. Structure and molecular-model refinement of Rhizomucor miehei triacylglyceride lipase—a case-study of the use of simulated annealing in partial model refinement. Acta Crystallogr B. 48: 307-319
Holmquist, M., Martinelle, M., Berglund, P., Clausen, I G., Patkar, S., Svendsen, A. Hult, K. 1993. Lipases from Rhizomucor miehei and Humicola lanuginosa: Modification of the lid covering the active site alters enantioselectivity. J Protein Chem. 12: 749-757
Khan, F I., Lan, D., Durrani, R., Huan, W., Zhao, Z., Wang. Y. 2017. The lid domain in lipases: Structural and functional determinant of enzymatic properties. Front Bioeng Biotech. 5
Lafaquière, V., Barbe, S., Puech-Guenot, S., Guieysse, D., Cortés, J., Monsan, P., Siméon, T., André, I., Remaud-Siméon, M. 2009. Control of Lipase Enantioselectivity by Engineering the Substrate Binding Site and Access Channel. Chem Bio Chem, 10: 2760-2771
Moroz, O. V., Blagova, E., Reiser, V., Saikia, R., Dalal, S., Jorgensen, C. I., Bhatia, V. K., Baunsgaard, L., Andersen, B., Svendsen, A., Wilson, K. S., Novel inhibitory function of the Rhizomucor miehei lipase propeptide and three-dimensional structures of its complexes with the enzyme. 2019. ACS Omega. 4: 9964-9975
Pinholt C., Fanø M., Wiberg C., Hostrup S., Bukrinsky J T., Frokjaer S., Norde W., Jorgensen L. Influence of glycosylation on the adsorption of Thermomyces lanuginosus lipase to hydrophobic and hydrophilic surfaces. 2010. Eur J Pharm Sci. 40(4): 273-81
Sehnal, D., Rose, A S., Kovca, J., Burley, S K., Velankar, S. 2018. Mol*: Towards a common library and tools for web molecular graphics MolVA/EuroVis Proceedings.
Wu, S. and Letchworth, G J. 2004. High efficiency transformation by electroporation of Pichia pastoris pretreated with lithium acetate and dithiothreitol. Biotechniques. 36(1): 152-154
Yedavalli, P. and Rao, N M. Engineering the loops in a lipase for stability in DMSO. 2013. Protein Eng Des Sel. 26(4): 317-324
Zhang, J., Lin, Y., Sun, Y., Ye, Y., Zheng, S., Han, S. 2012. High-throughput screening of B factor saturation mutated Rhizomucor miehei lipase thermostability based on synthetic reaction. Enzyme and Microbial Technology. 50 (6-7): 325-330

Sequences
SEQ ID No. 1:
WT RML
GAAGCCGAAGCTTCCATCGACGGAGGTATTAGAGCCGCTACT
TCTCAGGAAATCAACGAACTTACTTACTATACAACTTTGTCAGCT
AATTCTTACTGTAGAACTGTTATTCCTGGTGCTACTTGGGATTGC
ATACATTGTGACGCCACTGAAGATTTAAAGATAATTAAAACCTGG
TCTACTTTGATTTACGACACTAACGCTATGGTTGCTAGAGGAGAT
TCCGAGAAGACTATTTATATCGTGTTTAGAGGTTCTTCATCTATT
CGTAATTGGATCGCTGATTTGACATTCGTTCCAGTCTCTTACCCT
CCAGTTTCTGGTACTAAGGTTCACAAAGGATTTCTTGATTCTTAT
GGTGAAGTTCAAAACGAGTTGGTTGCTACTGTCTTGGATCAGTTT
AAACAATACCCATCTTATAAGGTTGCTGTCACTGGTCACTCTTTG
GGAGGTGCTACTGCCTTGCTGTGTGCTTTAGATTTATACCAGAGA
GAGGAAGGATTGTCTTCAAGTAACCTATTCTTGTACACTCAAGGT
CAGCCTAGAGTTGGAGATCCAGCATTTGCTAATTATGTGGTTTCT
ACTGGTATTCCATATAGACGTACTGTTAACGAAAGAGACATAGTA
CCACACTTGCCTCCAGCTGCCTTCGGATTTCTGCATGCCGGTGAA
GAGTACTGGATCACAGATAATTCTCCTGAAACCGTTCAAGTGTGT
ACATCTGATTTAGAGACTTCCGACTGCTCTAACAGTATTGTTCCA
TTTACTTCAGTTCTTGATCATTTGTCTTATTTTGGAATTAACACC
GGTTTGTGTACTTAA
SEQ ID No. 2:
WT RML
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPFTSVLDHLSYFGINTGLCT
SEQ ID No. 3:
RML237
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTNDTETSDCSNSIVPFTSVLDHLSYFGINTGLCT
SEQ ID No. 4:
RML243
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSNCTNSIVPFTSVLDHLSYFGINTGLCT
SEQ ID No. 5:
RML251
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPNTTVLDHLSYFGINTGLCT
SEQ ID No. 6:
RML 237 + 251
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTNDTETSDCSNSIVPNTTVLDHLSYFGINTGLCTS
ED ID No. 7:
RML 243 + 251
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSNCTNSIVPNTTVLDHLSYFGINTGLCT
SEQ ID No. 8:
RML F251A + S253T
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPATTVLDHLSYFGINTGLCT
SEQ ID No. 9:
RML F251D + S253T
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPDTTVLDHLSYFGINTGLCT
SEQ ID No. 10:
RML F251Q + S253T
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLECSDASNSIVPQTTVLDHLSYFGINTGLCT
SEQ ID No. 11:
RML F251N
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPNTSVLDHLSYFGINTGLCT
SEQ ID No. 12:
RML F251A
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLECSDASNSIVPATSVLDHLSYFGINTGLCT
SEQ ID No. 13:
RML F251D
(Mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPDTSVLDHLSYFGINTGLCT
SEQ ID No. 14:
RML F251Q
(Mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPQTSVLDHLSYFGINTGLCT
SEQ ID No. 15:
RML F251C
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPCTSVLDHLSYFGINTGLCT
SEQ ID No. 16:
RML F251E
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPETSVLDHLSYFGINTGLCT
SEQ ID No. 17:
RML F251G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPGTSVLDHLSYFGINTGLCT
SEQ ID No. 18:
RML F251H
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPHTSVLDHLSYFGINTGLCT
SEQ ID No. 19:
RML F251I
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPITSVLDHLSYFGINTGLCT
SEQ ID No. 20:
RML F251K
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPKTSVLDHLSYFGINTGLCT
SEQ ID No. 21:
RML F251L
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPLTSVLDHLSYFGINTGLCT
SEQ ID No. 22:
RML F251M
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPMTSVLDHLSYFGINTGLCT
SEQ ID No. 23:
RML F251P
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPPTSVLDHLSYFGINTGLCT
SEQ ID No. 24:
RML F251R
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPRTSVLDHLSYFGINTGLCT
SEQ ID No. 25:
RML F251S
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPSTSVLDHLSYFGINTGLCT
SEQ ID No. 26:
RML F251T
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPTTSVLDHLSYFGINTGLCT
SEQ ID No. 27:
RML F251V
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPVTSVLDHLSYFGINTGLCT
SEQ ID No. 28:
RML F251W
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPWTSVLDHLSYFGINTGLCT
SEQ ID No. 29:
RML F251Y
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPYTSVLDHLSYFGINTGLCT
SEQ ID No. 30:
RML I204A
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDAVPHLPPAAFGFLHAGEEYWIT
DNSPETVQVCTSDLETSDCSNSIVPFTSVLDHLSYFGINTGLCT
SEQ ID No. 31:
RML I204D
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDDVPHLPPAAFGFLHAGEEYWIT
DNSPETVQVCTSDLETSDCSNSIVPFTSVLDHLSYFGINTGLCT
SEQ ID No. 32:
RML I204F
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDFVPHLPPAAFGFLHAGEEYWIT
DNSPETVQVCTSDLETSDCSNSIVPFTSVLDHLSYFGINTGLCT
SEQ ID No. 33:
RML I204N
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDNVPHLPPAAFGFLHAGEEYWIT
DNSPETVQVCTSDLETSDCSNSIVPFTSVLDHLSYFGINTGLCT
SEQ ID No. 34:
RML I204R
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDRVPHLPPAAFGFLHAGEEYWIT
DNSPETVQVCTSDLETSDCSNSIVPFTSVLDHLSYFGINTGLCT
SEQ ID No. 35:
RML V254A
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPFTSALDHLSYFGINTGLCT
SEQ ID No. 36:
RML V254D
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPFTSDLDHLSYFGINTGLCT
SEQ ID No. 37:
RML V254F
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPFTSFLDHLSYFGINTGLCT
SEQ ID No. 38:
RML V254N
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPFTSNLDHLSYFGINTGLCT
SEQ ID No. 39:
RML V254R
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALDLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPFTSRLDHLSYFGINTGLCT
SEQ ID No. 40:
RML 251 + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPNTTVLDHLSYFGINTGLCT
SEQ ID No. 41:
RML F251N + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPNTSVLDHLSYFGINTGLCT
SEQ ID No. 42:
RML F251A + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLECSDASNSIVPATSVLDHLSYFGINTGLCT
SEQ ID No. 43:
RML F251D + D156G
(Mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPDTSVLDHLSYFGINTGLCT
SEQ ID No. 44:
RML F251Q + D156G
(Mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPQTSVLDHLSYFGINTGLCT
SEQ ID No. 45:
RML F251C + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPCTSVLDHLSYFGINTGLCT
SEQ ID No. 46:
RML F251E + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPETSVLDHLSYFGINTGLCT
SEQ ID No. 47:
RML F251G + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPGTSVLDHLSYFGINTGLCT
SEQ ID No. 48:
RML F251H + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPHTSVLDHLSYFGINTGLCT
SEQ ID No. 49:
RML F251I + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPITSVLDHLSYFGINTGLCT
SEQ ID No. 50:
RML F251K + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPKTSVLDHLSYFGINTGLCT
SEQ ID No. 51:
RML F251L + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPLTSVLDHLSYFGINTGLCT
SEQ ID No. 52:
RML F251M + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPMTSVLDHLSYFGINTGLCT
SEQ ID No. 53:
RML F251P + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPPTSVLDHLSYFGINTGLCT
SEQ ID No. 54:
RML F251R + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPRTSVLDHLSYFGINTGLCT
SEQ ID No. 55:
RML F251S + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPSTSVLDHLSYFGINTGLCT
SEQ ID No. 56:
RML F251T + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPTTSVLDHLSYFGINTGLCT
SEQ ID No. 57:
RML F251V + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPVTSVLDHLSYFGINTGLCT
SEQ ID No. 58:
RML F251W + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPWTSVLDHLSYFGINTGLCT
SEQ ID No. 59:
RML F251Y + D156G
(mature protein)
SIDGGIRAATSQEINELTYYTTLSANSYCRTVIPGATWDCIHCDA
TEDLKIIKTWSTLIYDTNAMVARGDSEKTIYIVFRGSSSIRNWIA
DLTFVPVSYPPVSGTKVHKGFLDSYGEVQNELVATVLDQFKQYPS
YKVAVTGHSLGGATALLCALGLYQREEGLSSSNLFLYTQGQPRVG
DPAFANYVVSTGIPYRRTVNERDIVPHLPPAAFGFLHAGEEYWIT
DNSPETVQVCTSDLETSDCSNSIVPYTSVLDHLSYFGINTGLCT

TABLE 6
List of primers used

| | Forward primer | Reverse Primer |
|---|---|---|
| AOX | GACTGGTTCCAATTGACAA | GCAAATGGCATTCTGACATCC |
| | CG (SEQ ID No. 60) | (SEQ ID No. 61) |
| RML237 | GTGTACAAACGATACTGAG | AAGTCTCAGTATCGTTTGTAC |
| | ACTTCCGACTGCTCTAA | ACACTTGAACGGTTTC (SEQ ID |
| | (SEQ ID No. 62) | No. 63) |
| RML243 | TTAGAGACTTCCAACTGCA | AGTGCAGTTGGAAGTCTCTAA |
| | CTAACAGTATTGTTCCATTT | ATCAGATGTAC (SEQ ID No. 65) |
| | (SEQ ID No. 64) | |
| RML251 | GTATTGTTCCAAACACTAC | GAACAGTAGTGTTTGGAACA |
| | TGTTCTTGATCATTTGTCT | ATACTGTTAGAGCAG |
| | (SEQ ID No. 66) | (SEQ ID No. 67) |
| RML | GTATTGTTCCAAACACTTC | GAACTGAAGTGTTTGGAACA |
| F251N | AGTTCTTGATCATTTGTCT | ATACTGTTAGAGCAGT |
| | (SEQ ID No. 68) | (SEQ ID No. 69) |
| RML | GTATTGTTCCAGCTACTTCA | GAACTGAAGTAGCTGGAACA |
| F251A | GTTCTTGATCATTTGTCT | ATACTGTTAGAGCAG |
| | (SEQ ID No. 70) | (SEQ ID No. 71) |
| RML | GTATTGTTCCAGATACTTCA | GAACTGAAGTATCTGGAACA |
| F251D | GTTCTTGATCATTTGTCT | ATACTGTTAGAGCAG |
| | (SEQ ID No. 72) | (SEQ ID No. 73) |
| RML | GTATTGTTCCACAAACTTC | GAACTGAAGTTTGTGGAACA |
| F251Q | AGTTCTTGATCATTTGTCT | ATACTGTTAGAGCAG |
| | (SEQ ID No. 74) | (SEQ ID No. 75) |
| RML | CGACTGCTCTAACAGTATT | TGGAACAATACTGTTAGAGCA |
| F251C | GTTCCATGTACTTCAGTTCT | GTCG (SEQ ID No. 77) |
| | TGATCATTTGTCT (SEQ ID | |
| | No. 76) | |
| RML | CGACTGCTCTAACAGTATT | TGGAACAATACTGTTAGAGCA |
| F251E | GTTCCAGAAACTTCAGTTC | GTCG (SEQ ID No. 77) |
| | TTGATCATTTGTCT (SEQ ID | |
| | No. 78) | |
| RML | CGACTGCTCTAACAGTATT | TGGAACAATACTGTTAGAGCA |
| F251G | GTTCCAGGTACTTCAGTTCT | GTCG (SEQ ID No. 77) |
| | TGATCATTTGTCT (SEQ ID | |
| | No. 79) | |
| RML | CGACTGCTCTAACAGTATT | TGGAACAATACTGTTAGAGCA |
| F251H | GTTCCACATACTTCAGTTCT | GTCG (SEQ ID No. 77) |
| | TGATCATTTGTCT (SEQ ID | |
| | No. 80) | |
| RML | CGACTGCTCTAACAGTATT | TGGAACAATACTGTTAGAGCA |
| F251I | GTTCCAATTACTTCAGTTCT | GTCG (SEQ ID No. 77) |
| | TGATCATTTGTCT (SEQ ID | |
| | No. 81) | |
| RML | CGACTGCTCTAACAGTATT | TGGAACAATACTGTTAGAGCA |
| F251K | GTTCCAAAGACTTCAGTTC | GTCG (SEQ ID No. 77) |
| | TTGATCATTTGTCT (SEQ ID | |
| | No. 82) | |
| RML | CGACTGCTCTAACAGTATT | TGGAACAATACTGTTAGAGCA |
| F251L | GTTCCATTGACTTCAGTTCT | GTCG (SEQ ID No. 77) |
| | TGATCATTTGTCT (SEQ ID | |
| | No. 83) | |
| RML | CGACTGCTCTAACAGTATT | TGGAACAATACTGTTAGAGCA |
| F251M | GTTCCAATGACTTCAGTTCT | GTCG (SEQ ID No. 77) |
| | TGATCATTTGTCT (SEQ ID | |
| | No. 84) | |
| RML | CGACTGCTCTAACAGTATT | TGGAACAATACTGTTAGAGCA |
| F251P | GTTCCACCAACTTCAGTTCT | GTCG (SEQ ID No. 77) |
| | TGATCATTTGTCT (SEQ ID | |
| | No. 85) | |
| RML | CGACTGCTCTAACAGTATT | TGGAACAATACTGTTAGAGCA |
| F251R | GTTCCAAGAACTTCAGTTC | GTCG (SEQ ID No. 77) |
| | TTGATCATTTGTCT (SEQ ID | |
| | No. 83) | |
| RML | CGACTGCTCTAACAGTATT | TGGAACAATACTGTTAGAGCA |
| F251S | GTTCCATCTACTTCAGTTCT | GTCG (SEQ ID No. 77) |
| | TGATCATTTGTCT (SEQ ID | |
| | No. 87) | |
| RML | CGACTGCTCTAACAGTATT | TGGAACAATACTGTTAGAGCA |
| F251T | GTTCCAACTACTTCAGTTCT | GTCG (SEQ ID No. 77) |
| | TGATCATTTGTCT (SEQ ID | |
| | No. 88) | |
| RML | CGACTGCTCTAACAGTATT | TGGAACAATACTGTTAGAGCA |
| F251V | GTTCCAGTTACTTCAGTTCT | GTCG (SEQ ID No. 77) |
| | TGATCATTTGTCT (SEQ ID | |
| | No. 89) | |
| RML | CGACTGCTCTAACAGTATT | TGGAACAATACTGTTAGAGCA |
| F251W | GTTCCATGGACTTCAGTTCT | GTCG (SEQ ID No. 77) |
| | TGATCATTTGTCT (SEQ ID | |
| | No. 90) | |
| RML | CGACTGCTCTAACAGTATT | TGGAACAATACTGTTAGAGCA |
| | GTTCCATACACTTCAGTTCT | |
| F251Y | TGATCATTTGTCT (SEQ ID | GTCG (SEQ ID No. 77) |
| | No. 91) | |
| RML | CGTACTGTTAACGAAAGAG | GTCTCTTTCGTTAACAGTACG |
| I204A | ACGCTGTACCACACTTGCC | TCTATATG (SEQ ID No. 93) |
| | TCCAGC (SEQ ID No. 92) | |
| RML | CGTACTGTTAACGAAAGAG | GTCTCTTTCGTTAACAGTACG |
| I204D | ACGATGTACCACACTTGCC | TCTATATG (SEQ ID No. 93) |
| | TCCAGC (SEQ ID No. 94) | |
| RML | CGTACTGTTAACGAAAGAG | GTCTCTTTCGTTAACAGTACG |
| I204F | ACTTTGTACCACACTTGCCT | TCTATATG (SEQ ID No. 93) |
| | CCAGC (SEQ ID No. 95) | |
| RML | CGTACTGTTAACGAAAGAG | GTCTCTTTCGTTAACAGTACG |
| I204N | ACAACGTACCACACTTGCC | TCTATATG (SEQ ID No. 93) |
| | TCCAGC (SEQ ID No. 96) | |
| RML | CGTACTGTTAACGAAAGAG | GTCTCTTTCGTTAACAGTACG |
| I204R | ACAGAGTACCACACTTGCC | TCTATATG (SEQ ID No. 93) |
| | TCCAGC (SEQ ID No. 97) | |
| RML | CTAACAGTATTGTTCCATTT | TGAAGTAAATGGAACAATAC |
| V254A | ACTTCAGCTCTTGATCATTT | TGTTAGAGC (SEQ ID No. 99) |
| | GTCTTATTTTGGAATTAAC | |
| | (SEQ ID No. 98) | |
| RML | CTAACAGTATTGTTCCATTT | TGAAGTAAATGGAACAATAC |
| V254D | ACTTCAGATCTTGATCATTT | TGTTAGAGC (SEQ ID No. 99) |
| | GTCTTATTTTGGAATTAAC | |
| | (SEQ ID No. 100) | |
| RML | CTAACAGTATTGTTCCATTT | TGAAGTAAATGGAACAATAC |
| V254F | ACTTCATTTCTTGATCATTT | TGTTAGAGC (SEQ ID No. 99) |
| | GTCTTATTTTGGAATTAAC | |
| | (SEQ ID No. 101) | |
| RML | CTAACAGTATTGTTCCATTT | TGAAGTAAATGGAACAATAC |
| V254N | ACTTCAAACCTTGATCATTT | TGTTAGAGC (SEQ ID No. 99) |
| | GTCTTATTTTGGAATTAAC | |
| | (SEQ ID No. 102) | |
| RML | CTAACAGTATTGTTCCATTT | TGAAGTAAATGGAACAATAC |
| V254R | ACTTCAAGACTTGATCATTT | TGTTAGAGC (SEQ ID No. 99) |
| | GTCTTATTTTGGAATTAAC | |
| | (SEQ ID No. 103) | |

The same reverse primers (SEQ ID Nos. 77. 93 and 99) may be used for the substitution of phenylalanine at position 251, isoleucine at position 204 and valine at position 254 as shown above.

1. A lipase variant comprising a first substituent of a lipase sequence of SEQ ID No. 2, wherein a position of the first substituent in SEQ ID No. 2 is selected from the group consisting of position 251, position 204, position 254, position 237, and position 243.

2. (canceled)

3. The lipase variant according to claim 1 wherein the position of the first substituent is at position 251 and the first substituent is selected from the group consisting of asparagine, alanine, cysteine, aspartic acid, glutamic acid, glycine, histidine, isoleucine, lysine, leucine, methionine, proline, glutamine, arginine, serine, threonine, valine, tryptophan and tyrosine.

4. (canceled)

5. The lipase variant according to claim 1 wherein the position of the first substituent is at position 204 and the first substituent is selected from the group consisting of phenylalanine, leucine, methionine, tyrosine, tryptophan, alanine, valine, glycine, methionine, proline, serine, threonine, asparagine, glutamine, aspartic acid, glutamic acid, arginine, histidine, and lysine.

6-9. (canceled)

10. The lipase variant according to claim 1 comprising a second substituent of the lipase sequence of SEQ ID No.2, wherein the position of the second substituent in SEQ ID No. 2 is position 253, wherein the first substituent is preferably at position 251.

11. The lipase variant according to claim 10 wherein the first substituent is at position 251 and selected from the group consisting of asparagine, alanine, aspartic acid, and glutamine.

12. The lipase variant according to claim 1 comprising a third substituent of the lipase sequence of SEQ ID No.2, wherein the position of the third substituent in SEQ ID No. 2 is position 156 wherein the first substituent is preferably at position 251.

13. The lipase variant according to claim 1 wherein the first substituent is at position 251, the lipase variant comprising a fourth substituent and a fifth substituent, wherein the fourth substituent and the fifth substituent is either at positions 237 and 239 respectively or positions 243 and 245 respectively.

14. The lipase variant according to claim 1 comprises a sequence selected from the group consisting of SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19. SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 56, SEQ ID No. 57, SEQ ID No. 58, and SEQ ID No. 59.

15. The lipase variant according to claim 1 comprising a sixth substituent of a lipase sequence of SEQ ID No. 2, wherein the first substituent and the sixth substituent are at or proximal to a surface loop region of the lipase sequence, wherein the first substituent is asparagine and the sixth substituent is threonine, the sixth substituent being two amino acid positions away from the first substituent.

16. The lipase variant according to claim 15 wherein the first substituent and sixth substituent is selected from the group consisting of S237N and L239T, D243N and S245T, and F251N and S253T.

17. A method comprising providing the lipase variant according to claim 1, a first ester and a reactant, wherein the reactant is selected from the group consisting of water, an acid and a second ester; and forming a reaction product between the first ester and the reactant with the aid of the lipase variant under suitable reaction conditions.

18. The method according to claim 17 wherein the first ester is a fatty acid ester, preferably a triacylglyceride.

19. (canceled)

20. The method according to claim 17 wherein the reactant is a medium chain fatty acid or ester.

21. (canceled)

22. The method according to claim 20 wherein the reaction product is a triacylglyceride with medium chain fatty acids at a 1,3-position of the triacylglyceride and a long chain fatty acid at a 2-position of the triacylglyceride.

23. The method according to claim 22 wherein the reactant is a long chain fatty acid or ester.

24. The method according to claim 17 wherein the first ester is palm oil and the reactant is oleic acid, oleic ester, linoleic acid or linoleic ester, and the first ester is palm fraction enriched in tripalmitin.

25. (canceled)

26. The method according to claim 17 wherein the fatty acid ester or triacylglyceride is selected from the group consisting of olive oil, high oleic sunflower oil, and high oleic rapeseed oil, and the reactant is stearic acid or stearic ester.

27. The method according to claim 26 wherein the reaction product is 1,3-distearoyl-2-oleoyl glycerol.

28. The method according to claim 17 wherein the lipase variant is SEQ ID No. 5.

29-31. (canceled)

32. The method according to claim 17 wherein the reactant is the second ester, and the reaction product is a transesterification product of the first ester and the second ester.

33. (canceled)
